(19) United States
(12) Patent Application Publication
(10) Pub. No.: US 2013/0046223 A1
(43) Pub. Date: Feb. 21, 2013

(54) Long Term Wound Dressing

(71) Applicant: Stephen Schrammel, West Allis, WI (US)

(72) Inventor: Stephen Schrammel, West Allis, WI (US)

(21) Appl. No.: 13/209,786

(22) Filed: Aug. 15, 2011

(51) Int. Cl. A61F 13/00; A61M 35/00

(52) U.S. Cl. 602/43; 604/305

(57) ABSTRACT

A long term wound dressing with a semi-permeable membrane containing a plurality of raised capillaries with internal capillary channels. A closable aperture, such as a slit, runs parallel to the capillary channels. The closable apertures open upon increased pressure caused by flushing fluid or other fluid between the dressing and the wound, allowing the fluid to drain away from the wound. The long term wound dressing may contain additional apertures for attaching flushing and drainage tubes.

### FIELD OF INVENTION

The present invention relates to the field of dressings for long term wounds, and more specifically the field of dressings that conform to a surface of a wound without facilitating growth of bacteria.

### BRIEF DESCRIPTION OF THE DRAWINGS

FIG. 1 illustrates a perspective view of an exemplary embodiment of a long term wound dressing.

FIG. 2 illustrates a top view of an exemplary embodiment of a long term wound dressing showing raised, squared capillaries.

FIG. 3 illustrates a bottom view of an exemplary embodiment of a long term wound dressing.

FIG. 4 illustrates a side view of an exemplary embodiment of a long term wound dressing.

FIG. 5 illustrates a cross-section of an exemplary capillary for a long term wound dressing.

FIG. 6 illustrates a magnified view of an exemplary closed cell for a long term wound dressing.

FIG. 7 illustrates a magnified view of an exemplary open cell for a long term wound dressing.

FIG. 8 illustrates the use of an exemplary embodiment of a long term wound dressing.

FIG. 9 illustrates an exemplary embodiment of a flanged tube.

### TERMS OF ART

As used herein, the term “capillary” refers to a fully or partially closed channel which facilitates the flow of a substance.

As used herein, the term “closable aperture” refers to an opening that may be fully or partially closed. A closable aperture may be usually closed but selectively opened upon the application or release of pressure.

As used herein, the term “drainage tube” means any structure or device used to drain fluid or other substances away from a wound.

As used herein, the term “flange” means a protuberance, ridge, rim or other structure adapted for mechanical fastening of a tube to capillaries.

As used herein, the term “flushing tube” means any structure or device used to apply fluid or other flushing substance between a dressing and a wound. Flushing tubes may use force to apply the fluid or other flushing substance.

As used herein, the term “integrally constructed” means constructed as a single unit.

As used herein, the term “interoperative structure” refers to a structure which helps stabilize and secure a drainage tube or flushing tube to a wound dressing. Interoperative structures may include, but are not limited to, flanges, adhesives, clips, pins, interlocking components and combinations thereof.

As used herein, the term “membrane” means any structure which forms a complete or partial physical barrier over a wound.

As used herein, the ter “membrane cell” means a section of a membrane all or partially defined or enclosed by capillaries.

As used herein, the term “semi-permeable” means selectively permeable, For example, a semi-permeable membrane may be permeable to molecules to oxygen, carbon dioxide, water vapor, and impermeable as to viruses and bacteria.

### BACKGROUND

Controlling the growth of bacteria on long term wounds is a difficult and tedious task. All of the conditions for an exuding wound to heal are the same conditions that enable the growth of bacteria. For example, a potentially fatal condition known as bed sores occurs from the growth of bacteria on exudate and fluids. The primary method known in the art for controlling the growth of bacteria on wounds is through the use of antiseptics and dressing changes.

A problem known in the art is that absorbent materials are used to conduct fluid away; however the absorbent materials themselves may then become a moist breeding ground for bacteria. Any void between the tissue (i.e., skin tissue) and the dressing risks exposure to harmful bacteria. The antibacterial and antiseptic solutions which are often used to control the growth of bacteria also kill or impede the growth of healthy skin cells, and it is undesirable to use them more than necessary. However, to prevent exposure of wounds to harmful bacteria, dressings must be changed approximately every four hours.

There are many problems known in the art with dressing changes. For example, each dressing change can damage fragile healing tissues, granulating tissues, and sloughy and necrotic wounds. Also, each dressing change risks exposure of an uncovered wound to new strains of bacteria transferred by airborne pathogens, bedding, or personnel. Dressing changes in hospitals require personnel supervision, which can be costly and similarly expose wounds to new strains of bacteria.

There is an unmet need for dressings which conform to a surface of a wound that do not become stagnant to facilitate growth of bacteria.

There is a further unmet need for dressings which do not require frequent dressing changes that disrupt the healing of tissues and potentially expose wounds to additional strains of bacteria.

There is a further unmet need for a dressing system which substantially limits the use of antibacterial and antiseptic solutions.

There is a further unmet need for a dressing system which does not use absorbent material which may facilitate growth of bacteria.

### SUMMARY OF THE INVENTION

The present invention is a long term wound dressing consisting of a semi-permeable membrane with a plurality of inter-connected capillaries with an inner capillary channel. The capillaries are raised off of the top surface of the membrane to create a honeycomb pattern on the membrane. A closable aperture runs parallel to the raised capillaries on the bottom surface of the membrane. When used as a wound dressing, the smooth side of the membrane is affixed against the wound. The increase in pressure between the wound and the dressing created during flushing causes the closable aperture to open and expose the inner channels of the capillaries, allowing flushing fluid to be drained away from the wound without requiring the dressing to be changed.

### DETAILED DESCRIPTION OF INVENTION

For the purpose of promoting an understanding of the present invention, references are made in the text to exemplary embodiments of long term wound dressing, only some of which are described herein. It should be understood that no limitations on the scope of the invention are intended by describing these exemplary embodiments. One of ordinary skill in the art will readily appreciate that alternate but functionally equivalent structures, devices and materials may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

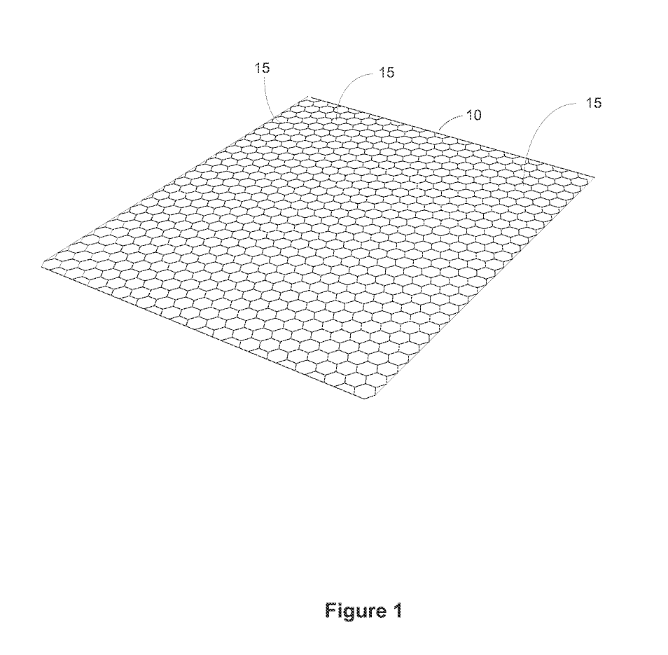

FIG. 1 illustrates a perspective view of an exemplary embodiment of long term wound dressing 100. Membrane 10 contains a plurality of membrane cells 15. In the exemplary embodiment shown, membrane cells 15 are arranged in a honeycomb pattern on membrane 10. However, in further exemplary embodiments, membrane cells 15 may be round, square, octagonal, triangular, angular, or any other shape or combination of shapes.

As illustrated in FIG. 1, membrane 10 is made of a basic soft silicone, such as platinum cure soft silicone, which is semi-permeable, or breathable. Membrane 10 is also stretchable up to 1000%. However, in further exemplary embodiments, membrane 10 may be made of any material known in the art which is medically acceptable for use as a long term wound dressing.

In the exemplary embodiment shown, each membrane cell 15 is approximately 2/10 of an inch from a flat side of membrane cell 15 to the directly opposite flat side. In further exemplary embodiments, membrane cells 15 may be consistently larger or smaller across membrane 10. In further exemplary embodiments, membrane cells 15 may be provided in various sizes on membrane 10.

As illustrated in FIG. 1, membrane 10 is a square sheet. In further exemplary embodiments, membrane 10 may be any shape or configuration which may be used to cover a wound. In still further exemplary embodiments, membrane 10 may be provided in a roll or other structure and selectively cut when needed to accommodate a specific wound or need.

In still further exemplary embodiments, membrane 10 may be specifically shaped to conform to a part of the body. For example, membrane 10 may be structured as a glove to be securely worn on a hand. In other exemplary embodiments, membrane 10 may be configured to cover a finger, toe, foot, leg, arm or other appendage. In further exemplary embodiments, membrane 10 may be a tube adapted to be secured around an arm, leg or other appendage.

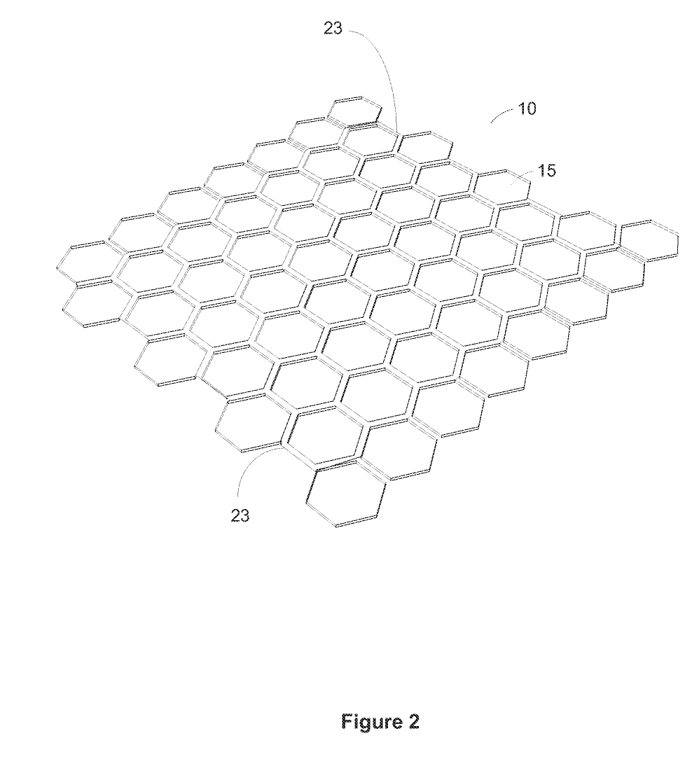

FIG. 2 illustrates a top view of an exemplary embodiment of long term wound dressing 100 showing membrane cells 15 bordered by raised capillaries 23. As illustrated in FIG. 2, raised capillaries 23 continuously boarder membrane cells 15. In further exemplary embodiments, raised capillaries 23 may border only some membrane cells 15 or may be configured in discontinuous sections.

The exemplary raised capillaries 23 illustrated in FIG. 2 are squared. In further exemplary embodiments, raised capillaries 23 may be rounded, pointed, or any other shape or combination of shapes.

As illustrated in FIG. 2, raise capillaries 23 are approximately 0.030 inches in height. In further exemplary embodiments, raised capillaries 23 may be approximately 0.010 to 0.050 inches in height.

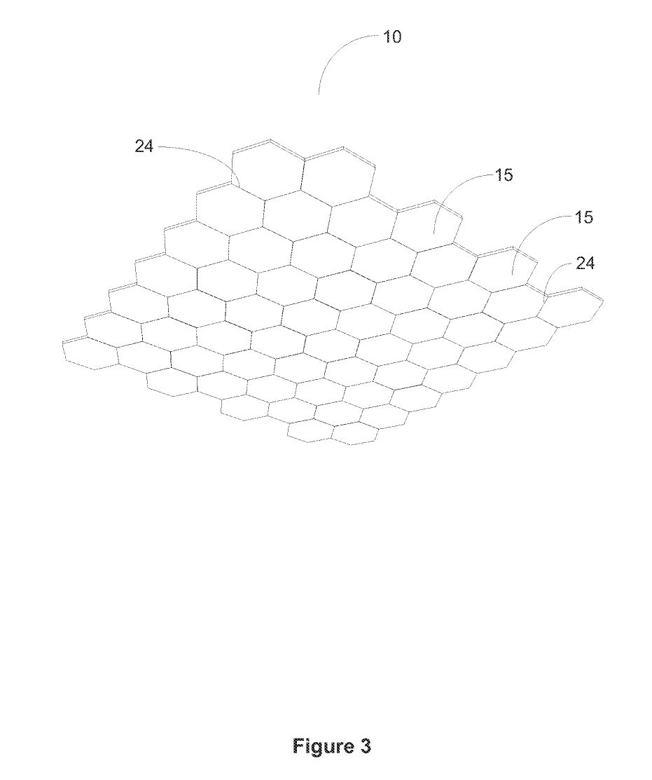

FIG. 3 illustrates a bottom view of an exemplary embodiment of long term wound dressing 100. As illustrated in FIG. 3, the bottom of membrane 10 is smooth with a single continuous membrane cell slit 24 running parallel to raised capillaries 23 (not shown) around membrane cells 15. When membrane 10 is stretched, membrane cells 15 are pulled away from each other and membrane cell slit 24 separates to expose capillary channel 25 (not shown).

In some exemplary embodiments, membrane cell slit 24 may not be a single continuous slit around membrane cells 15. For example, membrane 10 may contain multiple membrane cell slits in compartmentalized areas. In such exemplary embodiments, capillary channel 25 would not be continuous throughout membrane 10, but rather separated into multiple distinct channel systems.

This smooth side of membrane 10 is the side that would lay against a wound, with the side containing raised capillaries 23 facing away from the wound.

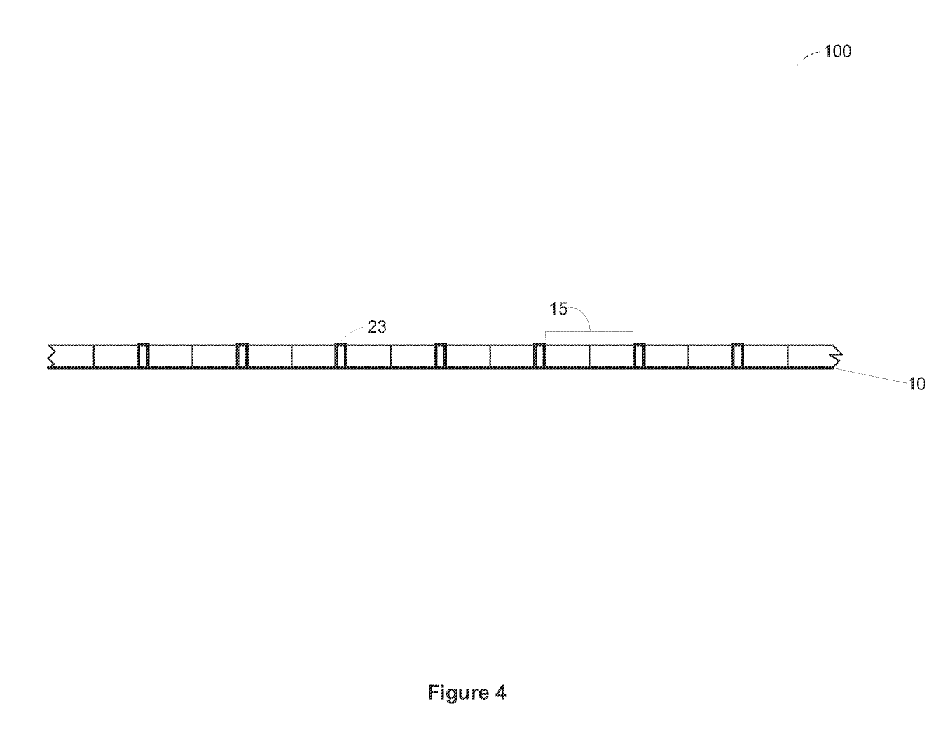

FIG. 4 illustrates a side view of an exemplary embodiment of a long term wound dressing 100. In the exemplary embodiment shown, membrane 10 is a single flat silicon sheet. Raise capillaries 23 are squared and boarder membrane cells 15.

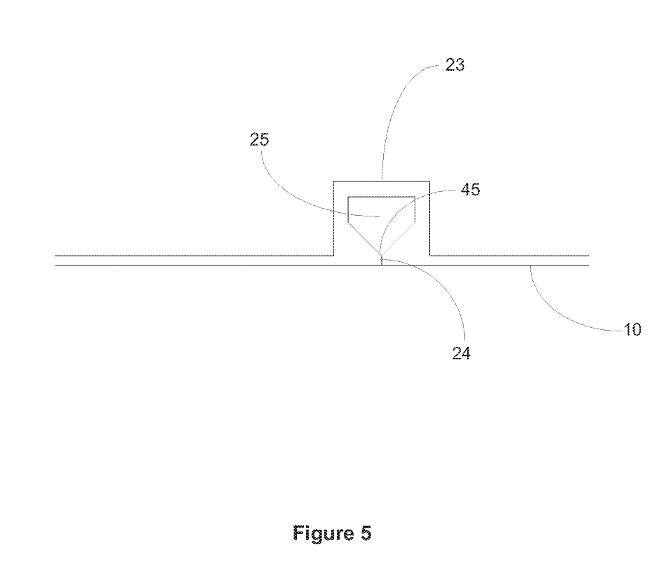

FIG. 5 illustrates a cross-section of raised capillary 23 showing capillary channel 25. In the exemplary embodiment shown, raised capillary 23 separates two membrane cells 15. Capillary channel 25 has an inverted house shape with the peak of the inverted house shape joining at membrane cell slit 24, creating connection point 45.

In further exemplary embodiments, capillary channel 25 may be triangular, squared, tub a angular, or any other shape which maintains a hollow channel and facilitates the flow of materials.

As illustrated in FIG. 5, capillary channel 25 is approximately 0.015 inches as measured from the flat top of capillary channel 25 to the peak at connection point 45. In further exemplary embodiments, capillary channel 25 may be larger or smaller to accommodate the flow of different materials through capillary channel 25.

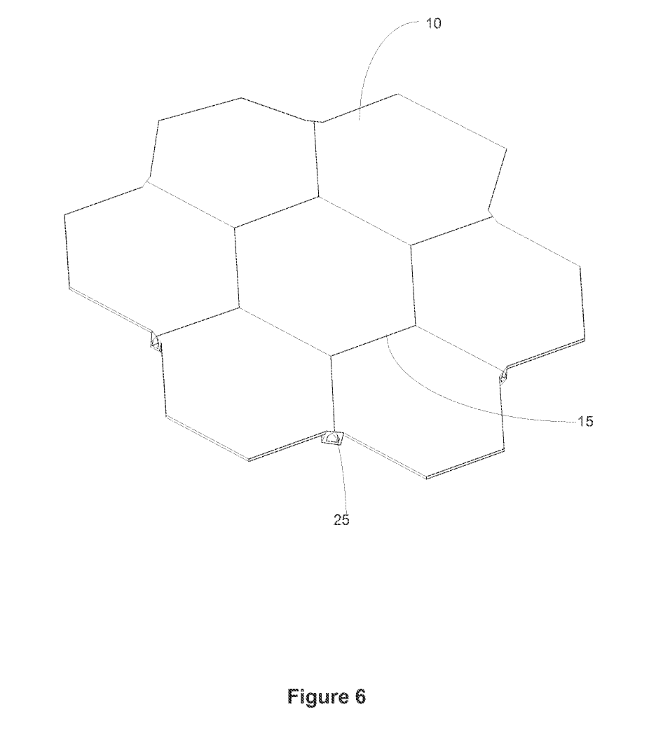

FIG. 6 illustrates a plurality of exemplary membrane cells 15 in a closed position. In the exemplary embodiment shown, membrane cells 15 are closed along membrane cell slit 24. Capillary channel 25 is closed.

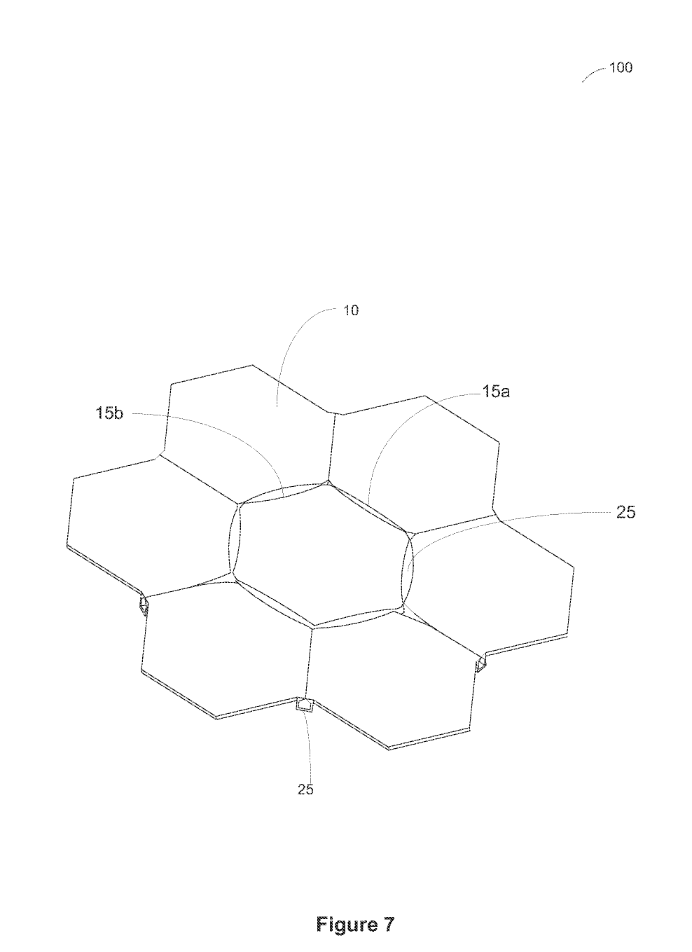

FIG. 7 illustrates a plurality of exemplary membrane cells 15 with capillary channel 25 open. In the exemplary embodiment shown, outer membrane cells 15a are being pulled away from center membrane cell 15b causing membrane cell slit 24 to separate and expose capillary channel 25.

When used as a dressing, the surface illustrated in FIG. 7 would lay against a wound. When the wound is flushed, the pressure of the flushing fluid between the wound and membrane 10 causes membrane 10 to stretch and membrane cell slit 24 to open an expose capillary channel 25. The flushing liquid is therefore provided with a path to carry it away from the wound and towards a drainage tube or other outlet. Other fluid build-up between the wound and membrane 10 may also be carried through capillary channel 25. In the exemplary embodiment shown, no absorbent material is used to draw moisture away from the wound.

In the exemplary embodiments described in FIGS. 1-7, long term wound dressing 100 is manufactured as a single piece by injection molding. In further exemplary embodiments, other molding methods, such as one-side molding or dip molding, may be used. In still further exemplary embodiments, long term wound dressing 100 membrane 10 and raised capillaries 23 may be separately manufactured and selectively or permanently attached. Membrane cell slits 24 may be cut on membrane 10 using a scalpel, knife, cutter or any other device known in the art.

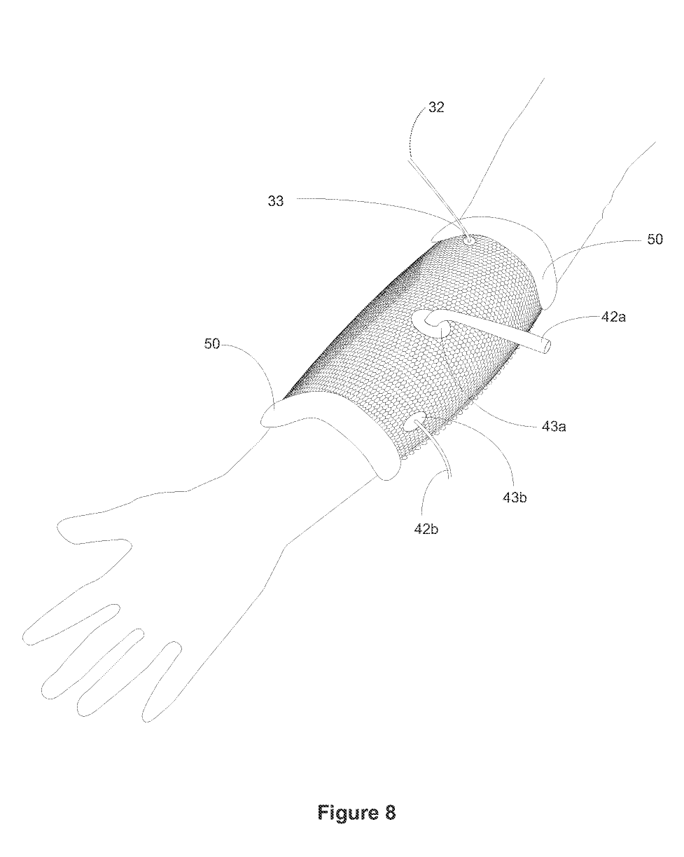

FIG. 8 illustrates long term wound dressing 100 in use on an arm. Membrane 10 is secured around its parameter by adhesive layer 50. In the exemplary embodiment shown, adhesive layer 50 is a viscous adhesive which blocks any outer open capillary channel 25 to create a liquid-tight seal, with medical tape securing membrane 10 to the arm. In further exemplary embodiments, any adhesive which creates a liquid tight seal around membrane 10 and secures membrane 10 to the arm or other body surface may be used.

As illustrated in FIG. 8, flushing tube 32 and drainage tubes 42a, 42b are secured to membrane 10. Rushing tube 32 applies flushing solution or other substance to the wound. Rushing solution makes its way to drainage tubes 42a, 42b using capillary channels 25 (not shown). In some exemplary embodiments, drainage tubes 42a, 42b may be connected to a receptacle, such as a bag, pouch or other container, collects the waste fluid to be discarded.

In some exemplary embodiments, more or fewer flushing or drainage tubes may be used. In still further exemplary embodiments, flushing tube 32 may be connected with an IV-type system, allowing a wound to be continuously irrigated and drained. For example, flushing tube 32 may be connected to a drip bag, fluid pump, negative pressure pump or other continuous fluid feed device.

As illustrated in FIG. 8, flushing tube 32 and drainage tubes 42a, 42b contain interoperative structures 33, 43a, and 43b respectively, to help secure flushing tube 32 and drainage tubes 42a, 42b to membrane 10. In the exemplary embodiment shown, interoperative structures 33, 43a and 43b are flanges which help prevent flushing tube 32 and drainage tubes 42a, 42b from disengaging membrane 10. In further exemplary embodiments, interoperative structures may be any structure or device known in the art which aids in securing flushing tube 32 and drainage tubes 42a, 42b to membrane 10, including, but not limited to, adhesives, pins, clips, interlocking structures and combinations of these structures and devices.

In some exemplary embodiments, interoperative structures 33, 43a and 43b may be or include an adhering interface, such as an adhesive layer, stitching or any other structural attachment component or combination of structural attachment components. In still further exemplary embodiments, interoperative structures 33, 43a and 43b may permanently or selectively secure flushing tube 32 and drainage tubes 42a, 42b to membrane 10.

As illustrated in FIG. 8, flushing tube 32 has a smaller diameter than drainage tube 42a. Drainage tubes generally have larger diameters than flushing tubes to allow particulate matter to escape with the draining fluids. In the exemplary embodiment shown, flushing tube 32 has a diameter of approximately ⅛ of an inch, with an internal bore being approximately 1/16 of an inch, while drainage tube 42a has a diameter of approximately ¼ of an inch with an internal bore of approximately 3/16 of an inch.

Drainage tube 42b is an example of a drainage tube which may be used during continuous irrigation and drainage of a wound. For example, when flushing tube 32 is connected with a continuous fluid feed device, such as a fluid bag similar to the type used with an IV, a small amount of fluid is continuously entering flushing tube 32. A smaller drainage tube, such as drainage tube 42b may then be used. However, drainage tube 42b may need to be completely flushed or replaced if it gets clogged.

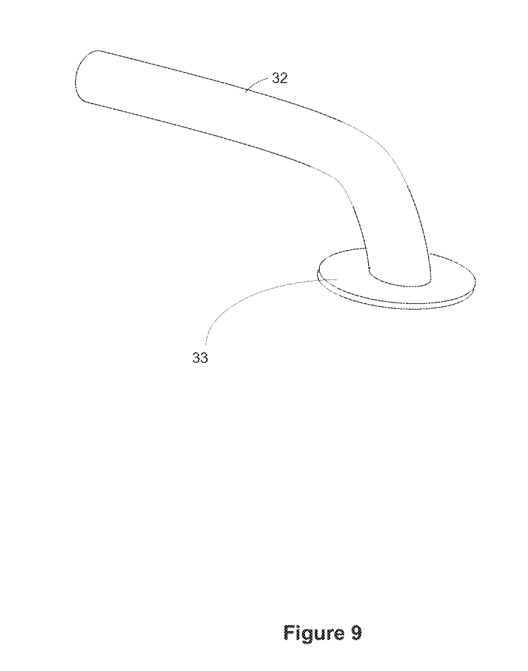

FIG. 9 illustrates an exemplary embodiment of a flushing or drainage tube using an interoperative structure which is a flange. In the exemplary embodiment shown, flushing or drainage tube 32 has flanged end 33. When used, flanged end 33 is secured against membrane 10 (not shown) to secure flushing or drainage tube 32.

In some exemplary embodiments, flushing or drainage tube 32 may be integrally assembled with a long term wound dressing 100 (not shown). In further exemplary embodiments, long term wound dressing 100 (not shown) may need to be selectively punctured in order to use flushing or drainage tube 32.

As illustrated in FIG. 10, flushing or drainage tube 32 is illustrated as a continuous length of cylindrical tubing. In further exemplary embodiments, flushing or drainage tube 32 may be squared, oblong or any shape which provides an inlet or outlet for fluids or other flushing or drainage substances.

In the exemplary embodiment shown, flushing or drainage tube 32 is a constant diameter. Flushing tubes usually have smaller diameters, while drainage tubes have larger diameters to allow debris, such as skin, scabs, and other particles, to drain a flushing fluid.

In some exemplary embodiments, flushing or drainage tube 32 may be collapsible.

What is claimed is:

1. A wound dressing apparatus comprised of:
at least one semi-permeable membrane;
said membrane further including a plurality of capillaries, each of said plurality of capillaries having an outer surface and an inner capillary channel through which a fluid may flow and wherein each of said plurality of capillaries intersects at a plurality of points to form a plurality of continuous capillaries and membrane cells surrounded by said plurality of capillaries; and
each of said membrane cells further includes at least one closable aperture.

2. The apparatus of claim 1 wherein each of said at least one closable aperture is a structural opening selected from a group consisting of a hole formed by a piercing tool, a slit, a flap, an aperture formed by molding, a perforation and combinations thereof.

3. The apparatus of claim 1 wherein said plurality of capillaries are integrally molded with said membrane.

4. The apparatus of claim 4 wherein said semi-permeable membrane and said plurality of capillaries are integrally constructed and of semi-permeable rubber.

5. The apparatus of claim 4 wherein said apparatus is constructed from a material selected from a group consisting of semi-permeable platinum cured soft silicone, latex, nitrile, butyl and EDPM.

6. The apparatus of claim 4 wherein said semi-permeable membrane and said plurality of capillaries are integrally constructed and of a semi-permeable synthetic rubber.

7. The apparatus of claim 1 wherein the cross section of said capillary has a shape selected from a group consisting of tubular, rounded, oval, squared, dome-shaped, house-shaped, inverted house-shaped, flattened or combinations thereof.

8. The apparatus of claim 1 which further includes at least one flushing tube.

9. The apparatus of claim 8 wherein each of said flushing tubes further includes at least one flange.

10. The apparatus of claim 8 wherein said at least one of said flushing tubes is a drainage tube.

11. The apparatus of claim 8 wherein at least two flushing tubes are drainage tubes.

12. The apparatus of claim 8 wherein said at least one flushing tube is collapsible.

13. The apparatus of claim 8 which further includes a layer of silicone adhesive to adhere to said at least one flushing tube to at least one of said plurality of capillaries.

14. The apparatus of claim 8 wherein said at least one flushing tube has an approximate ⅛^{th} inch diameter and an approximate 1/16^{th} inch bore relative to the size of said membrane cells.

15. The apparatus of claim 8 wherein said at least one flushing tube is a drainage tube with an approximate 3/16^{th} inch diameter relative to the size of said membrane cells, an approximate 3/16^{th} inch bore relative to the size of said membrane cells, and a collapsible exit.

16. The apparatus of claim 8 which further includes at least one collapsible tube that has a quarter inch diameter.

17. A wound dressing apparatus comprised of:
at least one semi-permeable membrane;
a plurality of capillaries integrally constructed with said semi-permeable membrane wherein said plurality of capillaries intersect at a plurality of points to form a plurality of continuous capillaries and membrane cells surrounded by said plurality of capillaries;
said capillaries being raised approximately 0.030 inches from said semi-permeable membrane relative to the size of said membrane cells and having an internal inverted-house shaped channel having a height of 0.015;
said semi-permeable membrane cells being honeycomb-shaped with six sides of equal length, wherein the distance from a first side to the side directly opposite said first side is approximately 2/10 of an inch; and
said semi-permeable membrane further including at least one closable aperture corresponding to at least one of said plurality of capillaries.

18. A wound dressing system comprised of:
at least one semi-permeable membrane;
said semi-permeable membrane further including a plurality of capillaries integrally constructed with said semi-permeable membrane, each of said plurality of capillaries having an outer surface and an inner capillary through which a fluid may flow wherein each of said plurality of capillaries intersects at a plurality of points to form a plurality of continuous capillaries and semi-permeable membrane cells surrounded by said plurality of capillaries;
a plurality of flushing tubes, wherein said at least one flushing tube is a drainage tube, wherein at least one flushing tube is an entry tube, wherein at least two flanged tubes are exit tubes;
at least one collection receptacle for receiving fluid from at least one of flushing tube; and
at least one continuous fluid feed device for introducing fluid into said at least one of said plurality of capillaries.

19. The system of claim 18 wherein said collection receptacle is a structure selected from a group consisting of a bag, a bulb, a vial and an absorptive material.

20. The system of claim 18 which further includes at least one inter-operative structure to connect the tubes to said semi-permeable membrane.

21. The system of claim 1 wherein said capillaries are interconnected inter-operatively to facilitate continuous flow of fluid through said capillaries.
